(19) United States
(12) Patent Application Publication
(10) Pub. No.: US 2015/0314047 A1
(43) Pub. Date: Nov. 5, 2015

(54) BORON COMPOSITE SURFACE COATINGS AND THEIR APPLICATION ON IMPLANTABLE DEVICES TO ACCELERATE OSSEOUS HEALING

(71) Applicant: Rutgers, The State University of New Jersey, New Brunswick, NJ (US)

(72) Inventors: Sheldon S. Lin, Chatham, NJ (US); David N. Paglia, New Britain, CT (US); James P. O'Connor, Fanwood, NJ (US); Eric Breitbart, South Orange, NJ (US); Joseph Benevenia, Montclair, NJ (US)

(73) Assignee: RUTGERS, THE STATE UNIVERSITY OF NEW JERSEY, New Brunswick, NJ (US)

(21) Appl. No.: 14/267,633

(22) Filed: May 1, 2014

Related U.S. Application Data
(63) Continuation of application No. 14/005,998, now abandoned.

(51) Int. Cl. A61L 31/08; A61L 31/16

(52) U.S. Cl. CPC A61L 31/088; A61L 31/16; A61L 2300/412; A61L 2300/102; A61L 2430/02

(57) ABSTRACT

The present invention discloses boron composite surface coatings, application of these coatings onto implantable devices, and use of the implantable devices for accelerating osseous healing. The implantable devices have wide applications, including but not limited to treating bone fracture, bone trauma, arthrodesis, and other bone deficit conditions, as well as bone injuries incurred in military and sports activities.

### CROSS-REFERENCE TO RELATED APPLICATIONS

This application claims priority under 35 U.S.C. §119(e) to U.S. Provisional Patent Application Ser. No. 61/454,061, filed on Mar. 18, 2011, which is hereby incorporated by reference in its entirety.

### FIELD OF THE INVENTION

The present invention relates to compositions comprising boron compounds, application of such boron composite surface coatings upon implantable devices, the implantable devices coated with such boron composite surface coatings, and methods of using these implantable devices for accelerating bone fracture or osseous healing.

### BACKGROUND OF THE INVENTION

Because up to 10% of the 6.2 million fractures sustained annually proceed to delayed union and non-union, (Praemer, A., et al., Amer. Acad. of Orthopaedic Surgeons, 85-124 (1992)), development of an ideal osseous adjunct is much needed, which would not only ameliorate significant military issues, but to a greater extent, meet clinical challenges throughout the United States.

Boron has been shown to regulate mineralized tissue formation in osteoblasts (Hakki, S., et al., J. Trace Elem. Med. Biol., 24(4): 243-250 (2003)), and dietary boron is beneficial for bone growth and maintenance and may enhance the strength of the axial skeleton in rats (Chapin, R., et al., Biol. Trace Elem. Res., 66(1-3): 395-399 (1998)). Hakki and colleagues detected increases in Bone Morphogenic Proteins (BMPs)-4, -6, and -7 for pre-osteoblastic cells at 0.1, 1, 10, and 100 ng/mL boron concentrations. RT-PCR results from this study demonstrated regulation in favor of osteoblastic function for Collagen type I (COL I), Osteopontin (OPN), Bone Sialoprotein (BSP), Osteocalcin (OCN) and RunX2 mRNA expressions for boron treatment groups in comparison with untreated control groups. Chapin et al demonstrated that animals administered with boron at several concentrations had 10% higher resistance to vertebral crushing force.

Boron is an essential element for appropriate bone healing. For example, Gorustovich et al. have shown that rats fed with boron deficient diets had lower levels of osteogenesis, following tooth extraction, compared to rats fed with 3 mg/kg daily boron diets (Gorustovich, A., et al., Anat. Rec. (Hoboken), 291(4): 441-447 (2008)). Research conducted by Benderdour and colleagues support this finding. Benderdour found that dietary boron deprivation in mice alters periodontal bone formation and remodeling (Benderdour, M., et al., J. Trace Elem. Med. Biol., 12(1): 2-7 (1998)).

A study by Koga et al has examined the toxicity of cubic boron nitride as a component for surgical cutting tools using human origin cultured cells (Koga, K., et al., Toxicol. In Vitro, 20(8): 1370-1377 (2006)). While cobalt negatively affected cell survival, including cell death, cubic boron nitride did not affect cell survival, even at reasonably high concentrations.

However, no evaluation of boron composite as a surface coating on orthopedic device on bone fracture healing or other bone regenerative processes has been performed in spite of the generally low toxicity of boron materials.

### SUMMARY OF THE INVENTION

The present invention provides boron composite surface coatings applicable on orthopedic devices and methods of using such coated devices for accelerating osseous healing or other bone regenerative processes. The methods can accelerate bone regeneration by stimulating insulin signaling at a fracture site.

In one aspect, the present invention provides boron composite surface coatings applied on an implantable device, said coating containing boron in the form of boron element or a boron-containing compound. In some embodiments, the boron element in the coating forms a composite with at least a metal element, preferably a transition metal atom. The boron-containing compound is preferably a transition metal boride.

In another aspect, the present invention provides application of boron composite surface coatings onto implantable devices.

In another aspect, the present invention provides implantable devices coated by boron composite surface coatings.

In another aspect, the present invention provides a method of promoting bone healing in a patient using implantable devices coated by boron composite surface coatings.

This invention, based on a novel concept, represents a significant paradigm shift from the present Orthopedic implant technology by providing unique boron-containing composite surface coatings applied upon Orthopedic devices. The methods of the present invention are applicable to devices including, but not limited to, plates, rods, screws, implants, arthroplasty implants or orthopedic devices utilized to stabilize fractures, osseous defects or tendon osseous junction, optionally in conjunction with the use of allograft/autograft or orthopedic biocomposite.

Surface modification of the orthopedic implants provides significant advantages including, but not limited to, ease of use, improved material properties (e.g., surface hardness), simple sterilization protocols, no need of special storage (i.e., refrigeration), and compatibility with the existing orthopedic devices, such as those made from titanium, zirconium, cobalt-chrome, stainless steel, or other specialty metals or their alloys. Accelerated bone regeneration can be achieved by coating the devices with a unique boron composite, whether the “devices” be plates, rods, screws, implants, arthroplasty implants or orthopedic devices utilized to stabilize fractures, osseous defects, to treat delayed union/non union, for allograft/autograft incorporation or tendon/liagment osseous junction in conjunction with the use of allograft/autograft or orthopedic biocomposite.

Optionally, and sometimes preferably, the method of the present invention is used in conjunction with local administration of a vanadium-based insulin-mimetic agent as disclosed in U.S. Provisional Application No. 61/295,234, filed Jan. 15, 2010, and PCT Application No. PCT/US11/21296, filed Jan. 14, 2011; and vanadium-based composite surface coatings as disclosed in U.S. Provisional Application Nos. 61/421,921, filed Dec. 10, 2010 and 61/428,342, filed Dec. 30, 2010, and PCT Application No. PCT/US11/62420, filed Dec. 9, 2011, all of which are hereby incorporated by reference in their entirety for all purposes.

### BRIEF DESCRIPTION OF THE DRAWINGS

FIG. 1 represents post-operative X-ray photographs taken immediately post-operative. (A) Einhorn model, (B) model used in this work. (Note in (B) the Kirschner wire is going through the trochanter, which helps to stabilize the fracture site and prevent migration of the Kirschner wire.)

FIG. 2 illustrates a Mechanical Testing Setup: (A) intact femur before embedded in ¾ inch square nut with Field's Metal, (B) intact femur embedded in hex nut and mounted in the mechanical testing apparatus, (C) intact femur mounted in the mechanical testing apparatus after torsional testing, (D) intact femur after torsional testing, (E) fractured femur after torsional testing showing spiral fracture indicative of healing, (F) fractured femur after torsional testing showing non-spiral fracture indicative of non-union.

### DETAILED DESCRIPTION OF THE INVENTION

The present invention is in part based on the discovery that boron composites as a surface coating on orthopedic devices can be used to accelerate bone regeneration by stimulating insulin signaling at a fracture site.

Current simple and comminuted fracture treatment relies upon restoring the bone's anatomy and stabilizing the fractured bone until the body is able to heal the fracture with newly produced bone. Adjuncts to this basic procedure, such as a method to significantly enhance bone regeneration while maintaining appropriate blood flow and preventing infection, have potential to revolutionize this field. Osseous agents such as boron or boron-containing compounds can enhance fracture callus strength by exploiting the healing responsiveness of insulin pathways. Localized therapy using this non-protein agent would minimize possibility of infection or other side effects or consequences that could be with systemic treatments.

Preliminary data has indicated that treatment using boron composite-coated implants is an effective method to treat fractures in non-diabetic patients. Mechanical parameters and microradiography revealed that bone has bridged within 4 weeks post fracture. Spiral fractures that occurred during mechanical testing reaffirm this phenomenon, which suggests local boron application at the dosages tested without a carrier may heal bone more than twice as rapidly as saline controls. This evidence opens up many potential applications for use of boron alone or being incorporated into a carrier as an alternative method for fracture healing.

In one aspect, the present invention provides a boron composite surface coating applied on an implantable device, the coating containing boron in the form of boron element or a boron-containing compound.

In one embodiment of this aspect, the present invention provides a boron composite surface coating, wherein the boron element forms a composite with at least one metal element.

In another embodiment of this aspect, the boron-containing compound contains boron and at least one transition metal.

In another embodiment of this aspect, the boron-containing compound is a transition metal boride.

In another embodiment of this aspect, the transition metal is selected from Ti, V, Cr, Mn, Fe, Co, Ni, Cu, Ta, Nb, Mo, Zr, and Re.

In another embodiment of this aspect, the boron-containing compound contains boron and at least one non-metal element selected from groups IVa-VIIa in the periodic table.

In another embodiment of this aspect, the at least one non-metal element is selected from the group consisting of O, C, N, and Si.

In another embodiment of this aspect, the boron-containing compound is selected from Fe₂B, FeB, Fe₃B, TiB₂, Ni₂B, ReB₂, Mn₄B, V₃B, CrB₂, AlB₂, SiB₃, and SiB₆.

In another aspect, the present invention provides use of a boron composite surface coating according to any one of the embodiments described herein for manufacture of an implantable device.

In another aspect, the present invention provides an implantable device coated by a boron composite surface coating.

In one embodiment of this aspect, the implantable device is coated by a boron composite surface coating, wherein the boron element forms a composite with at least one metal element.

In another embodiment of this aspect, the boron-containing compound contains boron and at least one transition metal.

In another embodiment of this aspect, the boron-containing compound is a transition metal boride.

In another embodiment of this aspect, the transition metal is selected from Ti, V, Cr, Mn, Fe, Co, Ni, Cu, Ta, Nb, Mo, Zr, and Re.

In another embodiment of this aspect, the boron-containing compound contains boron and at least one non-metal element selected from groups IVa-VIIa in the periodic table.

In another embodiment of this aspect, the at least one non-metal element is selected from the group consisting of O, C, N, and Si.

In another embodiment of this aspect, the boron-containing compound is selected from Fe₂B, FeB, Fe₃B, TiB₂, Ni₂B, ReB₂, Mn₄B, V₃B, CrB₂, AlB₂, SiB₃, and SiB₆.

In another embodiment of this aspect, the implantable device is selected from the group consisting of plates, rods, screws, implants, arthroplasty implants, and orthopedic devices.

In another embodiment of this aspect, the implantable device is a bone implant.

In another aspect, the present invention provides a method of promoting bone healing in a patient in need thereof, the method including treating the patient with an implantable device coated by a boron composite surface coating.

In one embodiment of this aspect, the composite surface coating applied onto the implantable device contains boron in the form of boron element or a boron-containing compound.

In another embodiment of this aspect, the boron element in the composite coating applied onto the implantable device forms a composite with at least one metal element.

In another embodiment of this aspect, the boron-containing compound in the composite coating applied onto the implantable device contains at least one transition metal.

In another embodiment of this aspect, the boron-containing compound in the composite coating applied onto the implantable device is a transition metal boride.

In another embodiment of this aspect, the transition metal in the composite coating applied onto the implantable device is selected from the group consisting of Ti, V, Cr, Mn, Fe, Co, Ni, Cu, Ta, Nb, Mo, Zr, and Re.

In another embodiment of this aspect, the boron-containing compound in the composite coating applied onto the implantable device contains at least one non-metal element selected from groups IVa-VIIa in the periodic table.

In another embodiment of this aspect, the at least one non-metal element of the boron-containing compound in the composite coating applied onto the implantable device is selected from the group consisting of O, C, N, and Si.

In another embodiment of this aspect, the boron-containing compound in the composite coating applied onto the implantable device is selected from the group consisting of Fe₂B, FeB, Fe₃B, TiB₂, Ni₂B, ReB₂, Mn₄B, V₃B, CrB₂, AlB₂, SiB₃, and SiB₆.

In another embodiment of this aspect, the implantable device is selected from the group consisting of plates, rods, screws, implants, arthroplasty implants, and orthopedic devices.

In another embodiment of this aspect, the implantable device is a bone implant.

In another embodiment of this aspect, the patient is afflicted with a bone condition selected from the group consisting of bone fracture, bone trauma, arthrodesis, and a bone deficit condition associated with post-traumatic bone surgery, post-prosthetic joint surgery, post-plastic bone surgery, post-dental surgery, bone chemotherapy treatment, congenital bone loss, post-traumatic bone loss, post-surgical bone loss, post-infectious bone loss, allograft incorporation or bone radiotherapy treatment.

In another embodiment of this aspect, the method is used in conjunction with administration of a cytotoxic agent, cytokine or growth inhibitory agent.

In another embodiment of this aspect, the method is used in conjunction with administration of a bioactive bone agent.

In another embodiment of this aspect, the bioactive bone agent is selected from the group consisting of peptide growth factors, anti-inflammatory factors, pro-inflammatory factors, inhibitors of apoptosis, MMP inhibitors, and bone catabolic antagonists.

In another embodiment of this aspect, the peptide growth factor is selected from the group consisting of IGF-1, IGF-2, PDGF (AA, AB, BB), BMPs, FGF (1 to 20), TGF-beta (1 to 3), aFGF, bFGF, EGF, VEGF, parathyroid hormone (PTH), and parathyroid hormone-related protein (PTHrP).

In another embodiment of this aspect, the anti-inflammatory factor is selected from the group consisting of anti-TNFα, soluble TNF receptors, ILlra, soluble IL1 receptors, IL4, IL-10, and IL-13.

In another embodiment of this aspect, the bone catabolic antagonist is selected from the group consisting of bisphosphonates, osteoprotegerin, and statins.

In another embodiment of this aspect, the method is used for treatment of fractures, osseous defects, delayed union or non-union, allograft/autograft incorporation or tendon/ligament osseous junction.

In another embodiment of this aspect, the method is used in conjunction with an allograft/autograft or orthopedic biocomposite.

In another embodiment of this aspect, the patient is a mammalian animal.

In another embodiment of this aspect, the patient is a human.

In another embodiment of this aspect, the patient is a non-diabetic human.

In another embodiment of this aspect, the patient is a horse or a dog.

Preferably, the boron composite surface coatings of the present invention are non-toxic, biologically compatible with blood and tissues in the patient, and the implantable devices coated by a boron composite surface coating according to the present invention have a hard, wear-resistant and corrosion-resistant surface. In particular, for a bone implant, it is particularly important to be wear-resistant and does not cause damages to bone tissues either chemically or physically, even when the bone is in motion.

One particular useful application of the present invention is, for example, in the treatment of military injuries involving bone fractures. Depending upon the level of energy, extremity fractures incurred in battle-related injuries may range from simple closed fracture to large segmental defects with a significant bone and soft tissue loss evident. Battle-related fractures have very high complication rates (47% in one study) with delayed union and non-union in 31% of all the fractures followed. (Pukljak, D., J. Trauma., 43(2): 275-282 (1997)). Many of these fractures occur in the extremities. Bullet wounds are often severe because a large amount of kinetic energy expends on the bone surface.

Using principles learned from previous wars and the development of Level I trauma centers, orthopedics care relies on the principles of timely restoration of anatomy, appropriate osseous stabilization, and subsequent restoration of function. Potential adjuncts to this basic concept through either mechanical (e.g., low intensity pulse ultrasound) or biological (e.g., growth factors like BMP-2) means can lead to acceleration of osseous healing for injured soldiers to have a faster recovery.

The high complication rate of severe military injuries with delayed union and non-unions parallels the observations seen in the civilian population who have risk factors for impaired bone healing. Risk factors include smoking, old age, steroid use, certain pharmaceuticals (i.e. anti-cancer drugs) and diabetes mellitus (DM). Clearly, if one is able to solve the impaired osseous healing associated with high-risk populations, one should be able to accelerate fracture healing in the normal, young, healthy soldiers with an insulin mimetic compound, such as local boron treatment. The present invention provides such a solution that would at least partially solve the problem.

The application of a unique boron composite surface coating upon orthopedic devices at the fracture site can have even wider scope of applications. For example, the unique boron composite surface coating upon orthopedic devices can find applications in treating both non-unions and delayed unions, for orthopedic use in trauma settings, and in sports medicine to treat a variety of fractures including fatigue fractures and acute sports-related fractures, such as acute fractures incurred during athletic activities as a result of overloading bone (boot top tibial fractures in skiing) or from ligament to tendon avulsion (tibial tubercle avulsion during long jumping). High school football injuries alone account for over 38,000 annual fractures. Sports fractures include, but are not limited to, tibial (49%), femoral (7%), and tarsal (25%) fractures which may differ depending on the individuals and causes of injury. (DeCoster, T., et al., “Sports fracture.” Iowa Orthopedic J., 14: 81-84 (1994)). The present work examined a mid-diaphyseal fracture pattern, but it is likely that other fracture patterns would heal in the same fashion.

The coatings of present invention can be formed by any methods known in the relevant art, for example, without limitation, those disclosed in Petrova, R. and Suwattananont, N., J. of Electronic Materials, 34(5): 8 (2005), which is hereby incorporated by reference. For example, suitable methods include chemical vapor deposition (CVD), physical vapor deposition (PVD), thermochemical treatment, oxidation, and plasma spraying. A suitable coating of the present invention may also contain combinations of multiple, preferably two or three, layers obtained by forming first boron diffusion coating followed by CVD (Zakhariev, Z., et al., Surf. Coating Technol., 31: 265 (1987)). Thermochemical treatment techniques have been well investigated and used widely in the industry. This is a method by which nonmetals or metals are penetrated by thermodiffusion followed by chemical reaction into the surface. By thermochemical treatment, the surface layer changes its composition, structure, and properties.

Other suitable coating techniques may include, but are not limited to, carburizing, nitriding, carbonitriding, chromizing, and aluminizing. Among these coating techniques, boronizing, being a thermochemical process, is used to produce hard and wear-resistant surfaces. Thermal diffusion treatments of boron compounds used to form iron borides typically require process temperatures of 700-1000° C. in either gaseous, solid, or salt media (Petrova, R. and Suwattananont, N., J. of Electronic Materials, 34(5): 8 (2005)). Boronizing is a process by which active boron atoms diffuse into the surface of substrate metal or alloy in order to produce a layer of borides. This treatment can be applied to ferrous materials, certain nonferrous materials such as titanium, tantalum, niobium, zirconium, molybdenum, nickel-based alloys, and cermets. Borides formed on steel surfaces increase their hardness (to about 2000 HV), wear resistance, and corrosion resistance (Wierzchon, T., Ed., Advances in Low-Temperature Plasma Chemistry, Technology, and Applications, Lancaster, Basel, Technomic Publishing Co. Inc. (1988); Hunger, H. and Trute, G., Heat Treatment Met., 21: 31 (1994); Pertek, A., Ed., Gas Boriding Condition for the Iron Borides Layers Formation, Materials Science Forum. Aedermannsdorf: Switzerland, Trans Tech Publications (1994); Venkataraman, B. and Sundararajan, G., Surf. Coating Technol., 73: 177 (1995); Xu, C., et al., J. Mater. Processing Technol., 65: 95 (1997)). Diffusion boronizing forms boride layers on metal and steel with good surface performance (Zakhariev, Z., et al., Less Common Metal, 117: 129-133 (1986); Wierzchon, T. and Belinski, P., Mater. Manufacturing Processing, 10: 121 (1995); Hunger, H., et al., Harterei technische Mittelungen, 52 (1997)). Other developments in boronizing include gas boronizing techniques such as fluidized bed boronizing and plasma boronizing. Physical vapor deposition and CVD, plasma spraying, and ion implantation are alternative non-thermochemical surface coating processes for the deposition of boron or co-deposition of boron and metallic elements onto a suitable metallic on nonmetallic substrate material.

As a person of ordinary skill in the art would appreciate, different coating techniques may be used to make the boron-based coatings and coated devices of the present invention in order to have desired properties suitable for specific purposes.

### Examples

### Materials and Methods

### The BB Wistar Rat Model

### Animal Source and Origin

Diabetic Resistance (DR) BB Wistar rats used in the study were obtained from a breeding colony at UMDNJ-New Jersey Medical School (NJMS). The rats were housed under controlled environmental conditions and fed ad libitum. All research protocols were approved by the Institutional Animal Care and Use Committee at University of Medicine and Dentistry of New Jersey-New Jersey Medical School.

### Diabetic Resistant BB Wistar Rats

A total of 9 DR BB Wistar rats were utilized in the study. Due to unstable fixation of mechanical testing, one sample was removed. The remaining 8 animals were used for mechanical testing, distributed amongst the control saline (n=5) and Boron coated rod (n=3) groups.

### Closed Femoral Fracture Model

Surgery was performed in DR animals between ages 80 and 120 days, using a closed mid-diaphyseal fracture model, on the right femur as described previously. (Beam, H. A., et al., J. Orthop. Res., 20(6): 1210-1216 (2002); Gandhi, A., et al., Bone, 38(4): 540-546 (2006)).

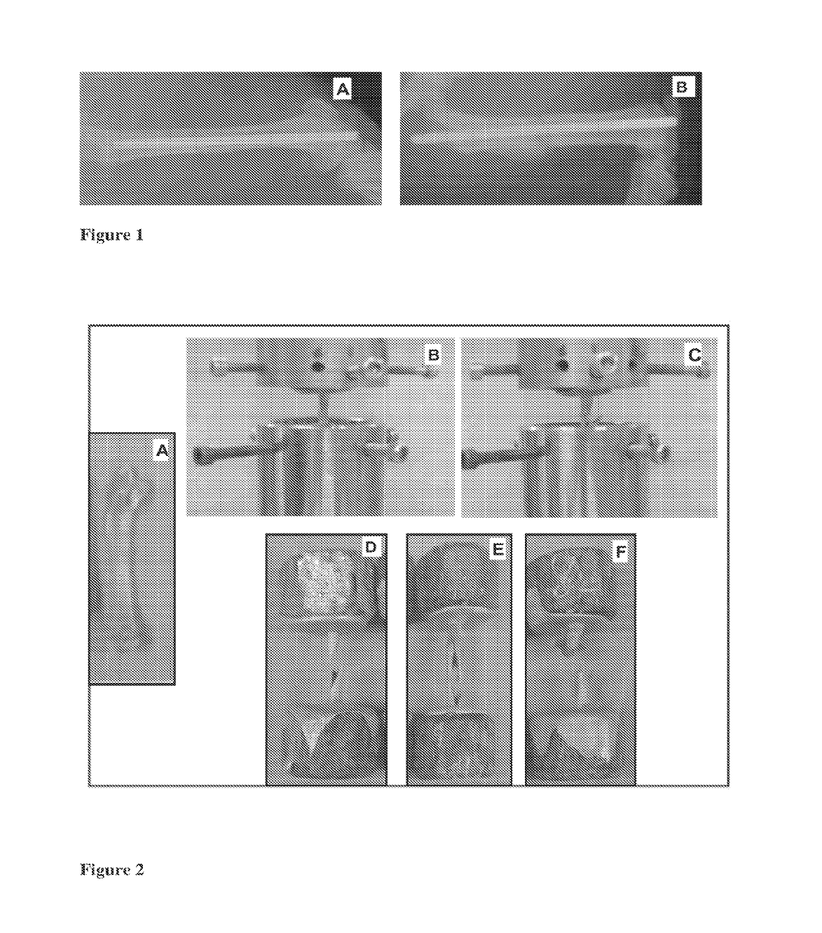

General anesthesia was administrated by intraperitoneal (IP) injection of ketamine (60 mg/kg) and xylazine (8 mg/Kg). The right leg of each rat was shaved and the incision site was cleansed with Betadine and 70% alcohol. An approximately 1 cm medial, parapatellar skin incision was made over the patella. The patella was dislocated laterally and the interchondylar notch of the distal femur was exposed. An entry hole was made with an 18 gauge needle and the femur was reamed with the 18 gauge needle. A Kirschner wire (316LVM stainless steel, 0.04 inch diameter, Small Parts, Inc., Miami Lakes, Fla.) which underwent thermochemical pack boriding was inserted the length of the medullary canal, and drilled through the trochanter of the femur. The Kirschner wire was cut flush with the femoral condyles. After irrigation, the wound was closed with 4-0 Vicryl resorbable suture. A closed mid-shaft fracture was then created unilaterally with the use of a three-point bending fracture machine. X-rays were taken to determine whether the fracture is of acceptable configuration. An appropriate fracture is an approximately mid-diaphyseal, low energy, transverse fracture (FIG. 1). The rats were allowed to ambulate freely immediately post-fracture. This closed fracture model is commonly used to evaluate the efficacy of osseous wound healing devices and drugs. (See, e.g., Nielsen, H. M., et al., Acta Orthop. Scand., 65(1): 37-41 (1994); Nakajima, F., et al. J. Orthop. Res., 19(5): 935-944 (2001); Beam, H. A., et al., J. Orthop. Res., 20(6): 1210-1216 (2002); Einhorn, T. A., et al., J. Bone Joint Surg. Am., 85-A(8): 1425-143.5 (2003); Schmidmaier, G., et al., Acta Orthop. Scand., 74(5): 604-610 (2003); Wildemann, B., et al., J. Biomed. Mater. Res., 65B(1): 150-156 (2003); Gandhi, A., et al., Bone 37(4): 482-490 (2005); Wang, H., et al., J. Orthop. Res., 23(3): 671-679 (2005); Gandhi, A., et al., Bone, 38(4): 540-546 (2006)).

### Experimental Treatments

### Orthopedic Device: IM Rod Pack Solid Bonding Technique

During boriding of steel and other metallic and alloy surfaces, boron atoms diffuse into the material and form various types of metal borides. In the case of ferrous alloys, most prominent borides are: Fe₂B and FeB. (Fe₃B may also form depending on the process parameters). Some of the boron atoms may dissolve in the structure interstitially without triggering any chemical reaction that can lead to boride formation. Iron borides (i.e., Fe₂B and FeB) are chemically stable and mechanically hard and hence can substantially increase the resistance of base alloys to corrosion, oxidation, and adhesive, erosive, or abrasive wear. Process conditions (such as duration of boriding, ambient temperature, type of substrate material and boriding media) may affect the chemistry and thickness of the borided surface layers. Due to the much harder nature of borided layers, boriding has the potential to replace some of the other surface treatment methods, such as carburizing, nitriding and nitrocarburizing.

Boride layers may achieve hardness values of more than 20 GPa depending on the chemical nature of the base materials. TiB₂ that forms on the surface of borided titanium substrates may achieve hardness values as high as 30 GPa; ReB₂ that forms on the surface of rhenium and its alloys may achieve hardness values as high as 50 GPa, while the hardness of boride layers forming on steel or iron-based alloys may vary between 14 GPa to 20 GPa. Such high hardness values provided by the boride layers are retained up to 650° C. Since there is no discrete or sharp interface between the boride layer and base material, adhesion strengths of boride layers to base metals are excellent. With the traditional methods mentioned above, boride layer thicknesses of up to 20 micrometer can be achieved after long periods of boriding time at much elevated temperatures. In addition to their excellent resistance to abrasive, erosive, and adhesive wear, the boride layers can also resist oxidation and corrosion even at fairly elevated temperatures and in highly acidic or saline aqueous media.

### Boron Composite Surface Coating Upon Orthopedic Devices: IM ROD Manufacturing

Annealed, cleaned, 1.6 mm Kirschner wire was packed in a boriding powder mixture contained within a 5 mm thick, heat resistant steel box. This allows the surfaces to be borided with a layer of about 10-20 micrometer thick. The mixture was composed of boron carbide, silicon carbide, and a boriding activator. The parts conformed to the container in which they were packed, and then were covered with a lid, which rests inside the container. This container was then weighted with an iron slug to ensure even trickling of the boriding agent during the manufacturing. The container was then heated to the boriding temperature as described in an electrically heated box with covered heating coils. The coated rods were allowed to come to room temperature and wiped with 95% ethyl alcohol prior to surgery.

### Microradiographic Evaluation

Serial microradiographs were obtained from all animals every two weeks post-surgery. Under the same anesthesia conditions as described previously, the rats were positioned prone so that lateral and anteroposterior (AP) views of their femurs could be obtained. Radiographs were taken using a Hewlett-Packard Faxitron (Model 43804-Radiographic Inspection System) and Kodak MinR-2000 mammography film. Exposures were performed for 30 seconds at 55 kVp. Additionally, magnified radiographs were obtained after the femurs were removed from the animals post-sacrifice. Qualitative analysis was performed on all radiographic samples. Two independent observers individually scored radiographs based on endosteal and cortical bridging on both lateral and AP femoral orientations. Averages amongst samples of the same group were computed to determine overall percentages of endosteal and cortical healing at 4 weeks.

All analysis was conducted in a blinded fashion using a five-point radiographic scoring system, 0=partial callus formation, 1=definite callus with bony union on one cortex, 2=definite callus with bony union on two cortices, 3=definite callus with bony union on two cortices, and 4=definite callus with bony union on all four cortices.

### Mechanical Testing

Fractured and contralateral femora were resected 4 weeks post-fracture. Femora were cleaned of soft tissue and the intramedullary rod was removed. Samples were wrapped in saline (0.9% NaCl) soaked gauze and stored at −20° C. Prior to testing, all femora were removed from the freezer and allowed to thaw to room temperature for three to four hours. The proximal and distal ends of the fractured and contralateral femora were embedded in ¾ inch square nuts with Field's Metal, leaving an approximate gauge length of 12 mm (FIG. 2). After measuring callus and femur dimensions, torsional testing was conducted using a servohydraulics machine (MTS Systems Corp., Eden Prairie, Minn.) with a 20 Nm reaction torque cell (Interface, Scottsdale, Ariz.) and tested to failure at a rate of 2.0 deg/sec. The maximum torque to failure and angle to failure were determined from the force to angular displacement data.

Peak torque to failure (Tₘₐₓ), torsional rigidity (TR), shear modulus (SM), and maximum torsional shear stress (SS) were calculated through standard equations. (Ekeland, A., et al., Acta Orthop. Scand., 52(6): 605-613 (1981); Engesaeter, L. B., et al., Acta Orthop. Scand., 49(6): 512-518 (1978); Beam, H. A., et al., J. Orthop. Res., 20(6): 1210-1216 (2002)). Tₘₐₓ and TR are considerd extrinsic properties while SM and SS are considered intrinsic properties. Tₘₐₓ was defined as the point where an increase in angular displacement failed to produce any further increase in torque. TR is a function of the torque to failure, gauge length (distance of the exposed femur between the embedded proximal and distal end) and angular displacement. SS is a function of the torque to failure, maximum radius within the mid-diaphyseal region and the polar moment of inertia. The polar moment of inertia was calculated by modeling the femur as a hollow ellipse. Engesaeter et al. demonstrated that the calculated polar moment of inertia using the hollow ellipse model differed from the measured polar moment of inertia by only 2 percent.

In order to compare the biomechanical parameters between different groups, the data was normalized by dividing each fractured femur value by its corresponding intact, contralateral femur value. Normalization was used to minimize biological variability due to differences in age and weight among rats.

In addition to the biomechanical parameters determined through torsional testing, the mode of failure can also provide substantial information. The mode of torsional failure as determined by gross inspection provided an indication as to the extent of healing. A spiral failure in the mid-diaphyseal region indicated a complete union, while a transverse failure through the fracture site indicated a nonunion. A combination spiral/transverse failure indicated a partial union (FIG. 2).

### Data and Statistical Analysis

Analysis of variance (ANOVA) was performed followed by Holm-Sidak post-hoc tests to determine differences (SigmaStat 3.0, SPSS Inc., Chicago, Ill.). A P value less than 0.05 was considered statistically significant.

### Results

### General Health

In this biomechanical experiment, animals among treatment groups were age matched. Blood glucose levels and age at surgery showed a significant difference between the Boron coated and saline groups (Table 1); however, the clinical relevance of this observation is difficult to ascertain since this range is within the normoglycemic value of Non-DM rats. These fluctuations may be a result of the small sample size and variations based on diet.

All animals were grouped within the same age within 40 days (80-120 days) and the difference between the average ages between these two groups was less than 10 days. Such a small age difference within this phase is unlikely to produce any major changes in healing rates.

TABLE 1
General Health of Non-DM BB Wistar Rats: Boron Surface Coating
(MechanicalTesting)

| | Blood Glucose | | |
|---|---|---|---|
| | (mg/dl)* | Age | |
| | Pre-Surgery | at Surgery | % Weight gain |
| Saline | 81.7 ± 4.3 | 99.0 ± 1.0 | 3.5 ± 2.3 |
| (n = 5) | | | |
| Boron | 94.7 ± 5.5 | 88.0 ± 0.0 | 15.3 ± 8.14 |
| Coated | | | |
| (n = 3) | | | |

The data represents average values ±standard deviation

### Mechanical Testing Results

The effect of therapy on healing of femur fractures by boron-coated implant in normal (non-diabetic) rats was measured by torsional mechanical testing. At the fourth week post-fracture, rats treated with boron displayed improved mechanical properties of the fractured femora compared to the untreated group. The shear modulus (Saline group vs. coated Boron rod group P<0.05), and maximum shear stress (Saline group vs. Boron rod group P<0.05), were both significantly increased compared in the Boron rod group when compared to the untreated group (Table 2). When the mechanical parameters of the fractured femora were normalized to the intact, contralateral femora, percent peak torque (Saline group vs. coated Boron rod group P<0.05, Saline group vs. Boron rod group P<0.05), torsional rigidity (Saline group vs. coated Boron rod group P<0.05), shear modulus (Saline group vs. coated Boron rod group P<0.05), and shear stress (Saline group vs. coated Boron rod group P<0.05) were all significantly greater in the local boron treated groups when compared to the saline group (Table 2).

To the best of our knowledge this is the first study to examine the effect of local boron treatment on fracture healing, quantified by mechanical testing. Our study demonstrated that local Boron bound to IM rods significantly improved the biomechanical parameters of fracture healing in non-diabetic animals. An earlier study examining the effect of boron surface coating on mechanical strength of bone in non-diabetic and diabetic animals revealed that boron had no effect on bone homeostasis in non-diabetic animals (Facchini, D. M., et al., Bone, 38(3): 368-377 (2006)). The fracture healing pathway is different than the bone homeostasis pathway. This is likely the primary reason for conflicting results presented in both models. Other possibilities include different dosages and delivery methods in each study.

TABLE 2
Four weeks Post-fracture mechanical testing with Boron ^{†}
Fractured femur values

| | Maximum | Maximum | | Maximum |
|---|---|---|---|---|
| | Torque to | Torsional | Shear | Shear |
| | failure | Rigidity | Modulus | Stress |
| | (Nmm) | (Nmm²/rad) | (MPa) | (MPa) |
| Saline (n = 5) | 178 ± 38 | 9,363 ± 5,032 | 235 ± 102 | 19 ± 3 |
| Boron Coated | 251 ± 93 | 19,683 ± 9,207 | 1,909 ± 1,582 * | 70 ± 46 * |
| (n = 3) | | | | |

Fractured femur values normalized to the contralateral (intact) femur

| | Percent | Percent | | Percent |
|---|---|---|---|---|
| | maximum | maximum | Percent | maximum |
| | torque to | torsional | shear | shear |
| | failure | rigidity | modulus | stress |
| Saline (n = 5) | 30 ± 18 | 19 ± 11 | 4 ± 2 | 11 ± 5 |
| Boron Coated | 68 ± 22 * | 73 ± 36 * | 23 ± 18 * | 33 ± 16 * |
| (n = 3) | | | | |

^{†} The data represents average values ± standard deviation
* Represents values significantly greater than the saline control group; p < 0.05

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the spirit and script of the invention, and all such variations are intended to be included within the scope of the following claims.

All references cited hereby are incorporated by reference in their entirety.

1. A boron composite surface coating applied on an implantable device, said coating comprising boron in the form of boron element or a boron-containing compound.

2. (canceled)

3. (canceled)

4. (canceled)

5. (canceled)

6. (canceled)

7. (canceled)

8. (canceled)

9. (canceled)

10. An implantable device coated by a boron composite surface coating.

11. The implantable device of claim 10, wherein said composite surface coating comprises boron in the form of boron element or a boron-containing compound.

12. (canceled)

13. (canceled)

14. A method of promoting bone healing in a patient in need thereof comprising treating said patient with an implantable device coated by a boron composite surface coating.

15. The method of claim 14, wherein said composite surface coating comprises boron in the form of boron element or a boron-containing compound.

16. The method of claim 15, wherein said boron element forms a composite with at least one metal.

17. The method of claim 15, wherein said boron-containing compound comprises at least one transition metal.

18. The method of claim 15, wherein said boron-containing compound is a transition metal boride.

19. The method of claim 17, wherein said transition metal is selected from Ti, V, Cr, Mn, Fe, Co, Ni, Cu, Ta, Nb, Mo, Zr, and Re.

20. The method of claim 14, wherein said boron-containing compound comprises at least one non-metal element selected from groups IVa-VIIa in the periodic table.

21. The method of claim 20, wherein said at least one non-metal element is selected from the group consisting of O, C, N, and Si.

22. The method of claim 15, wherein said boron-containing compound is selected from Fe₂B, FeB, Fe₃B, TiB₂, Ni₂B, ReB₂, Mn₄B, V₃B, CrB₂, AlB₂, SiB₃, and SiB₆.

23. The method of claim 14, wherein the implantable device is selected from the group consisting of plates, rods, screws, implants, arthroplasty implants, and orthopedic devices.

24. The method of claim 14, wherein the implantable device is a bone implant.

25. The method of claim 14, wherein said patient is afflicted with a bone condition selected from the group consisting of bone fractures, bone traumas, arthrodesis, and bone deficit conditions associated with post-traumatic bone surgery, post-prosthetic joint surgery, post-plastic bone surgery, post-dental surgery, bone chemotherapy treatment, congenital bone loss, post-traumatic bone loss, post-surgical bone loss, post-infectious bone loss, allograft incorporation or bone radiotherapy treatment.

26. (canceled)

27. The method of claim 14, wherein the method is used in conjunction with administration of a bioactive bone agent, cytotoxic agent, cytokine, or growth inhibitory agent.

28. The method of claim 27, wherein said bioactive bone agent is selected from the group consisting of peptide growth factors, anti-inflammatory factors, pro-inflammatory factors, inhibitors of apoptosis, MMP inhibitors, and bone catabolic antagonists.

29. The method of claim 28, wherein said peptide growth factor is selected from the group consisting of IGF-1, IGF-2, PDGF (AA, AB, BB), BMPs, FGF (1 to 20), TGF-beta (1 to 3), aFGF, bFGF, EGF, VEGF, parathyroid hormone (PTH), and parathyroid hormone-related protein (PTHrP): said anti-inflammatory factor is selected from the group consisting of anti-TNFα, soluble TNF receptors, IL1ra, soluble IL1 receptors, IL4, IL-10, and IL-13; and said bone catabolic antagonist is selected from the group consisting of bisphosphonates, osteoproteerin, and statins.

30. (canceled)

31. (canceled)

32. The method of claim 14, wherein the method is used for treatment of fractures, osseous defects, delayed union or non-union, allograft/autograft incorporation or tendon/ligament osseous junction.

33. The method of claim 32, wherein the method is used in conjunction with an allograft/autograft or orthopedic biocomposite.

34. (canceled)

35. (canceled)

36. (canceled)

37. (canceled)
